(19) United States
(12) Patent Application Publication
(10) Pub. No.: US 2022/0274098 A1
(43) Pub. Date: Sep. 1, 2022

(54) METHODS FOR PRODUCING STEP DIENES

(71) Applicants: EXXONMOBIL CHEMICAL PATENTS INC., Baytown, TX (US); THE TRUSTEES OF PRINCETON UNIVERSITY, Princeton, NJ (US)

(72) Inventors: ALEX E. CARPENTER, SEABROOK, TX (US); TZU-PIN LIN, SEABROOK, TX (US); IRENE C. CAI, WEBSTER, TX (US); GURSU CULCU, HUMBLE, TX (US); DANIELLE SINGLETON, HOUSTON, TX (US); PAUL J. CHIRIK, PRINCETON, NJ (US); C. ROSE KENNEDY, ROCHESTER, NY (US); MEGAN MOHADJER BEROMI, PASADENA, MD (US)

(73) Assignees: EXXONMOBIL CHEMICAL PATENTS INC., BAYTOWN, TX (US); THE TRUSTEES OF PRINCETON UNIVERSITY, PRINCETON, NJ (US)

(21) Appl. No.: 17/680,556

(22) Filed: Feb. 25, 2022

Related U.S. Application Data
(60) Provisional application No. 63/154,043, filed on Feb. 26, 2021.

(51) Int. Cl. B01J 37/04; C07C 2/30

(52) U.S. Cl. CPC B01J 37/04; C07C 2/30

(57) ABSTRACT

Methods for the hydroalkenylation of conjugated, 1,3-dienes using a diimine catalyst. The method comprises mixing a diene having at least five carbon atoms and an iron diimine complex at a temperature of about −60° C. to about 23° C. to provide a catalyst solution; and introducing one or more alpha olefins at a pressure of at least 300 psig to obtain a product comprising the substituted diene monomer.

### FIELD OF THE INVENTION

This invention relates to methods for the hydroalkenylation of conjugated dienes to give non-conjugated dienes. More particularly, this invention relates to methods for making 4-substituted hexadiene using diimine metal catalyst complexes.

### BACKGROUND OF THE INVENTION

There is a need to develop a broad array of curable elastomeric and curable plastic products derived from ethylene and other low-cost olefin precursors. For good cure performance, it is useful to employ at least one diene monomer in co-polymerizations, however, most widely used diene co-monomers (e.g., butadiene, isoprene, vinylnorbornene, ethylidene norbornene, 1,4-hexadiene, cyclopentadiene, dicylclopentadiene) suffer from various deficiencies. Dienes with 1,3 conjugation and sterically unencumbered dienes (i.e. 1,4-hexadiene), for example, are challenging to polymerize with good regioselectivity and productivity. Such sterically unencumbered dienes (i.e. 1,4-hexadiene) also suffer from poor cure performance, especially compared to cyclic dienes (i.e. 5-ethylidene-2-norbornene). Cyclic dienes are costly, complex to manufacture and exhibit unwanted effects relative to polymer glass transition temperatures.

An iron diimine complex stabilized by 1,5-cyclooctadiene has been used to hydrovinylate conjugated dienes using ethylene, as described in Schmidt, V. A. et al. (2018) “Selective [1,4]-Hydrovinylation of 1,3-Dienes with Unactivated Olefins Enabled by Iron Diimine Catalysts” J. Am. Chem. Soc., v. 140, pp. 3443-3453. However, generation of the iron diimine catalyst is performed using ethereal solvent at liquid nitrogen temperatures.

An iron diimine complex has been combined with a magnesium hydrocarbyl reagent at −50° C. in the presence of butadiene as described in Lee, H. et al. (2016) “Mechanistic Insight Into High-Spin Iron(I)-Catalyzed Butadiene Dimerization” Organometallics, v. 35, pp. 2923-2929. However, this work only describes the use of this catalyst system for the formation of 1,5-cyclooctadiene.

There is still a need for more suitable diene monomers that are cost-effective to manufacture and readily polymerized, so as to produce high diene content polymer at lower cost. The ability to produce substituted hexadiene as a compositionally pure monomer in a cost-effective manner allows for the commoditization of this diene in conjunction with single-site polymerization technology. It is therefore an object of the present invention to provide an improved method for making 4-substituted hexadiene monomer.

### SUMMARY OF THE INVENTION

Methods for the hydroalkenylation of conjugated, 1,3-dienes using a diimine transition metal catalyst are provided. In certain embodiments, the method comprises mixing a diene having at least five carbon atoms and a diimine metal complex to provide a catalyst solution and then introducing one or more alpha olefins to the catalyst solution to obtain a product comprising the substituted diene monomer. The substituted diene monomer is preferably 4-substituted hexadiene.

### BRIEF DESCRIPTION OF THE FIGURES

FIG. 1 (FIG. 1) depicts the ¹H NMR spectroscopic data of the product obtained in Example 1, according to one or more embodiments provided herein.

FIG. 2 (FIG. 2) depicts a focused spectrum of the ¹H NMR data depicted in FIG. 1.

FIG. 3 (FIG. 3) depicts another focused spectrum of the ¹H NMR data depicted in FIG. 1.

FIG. 4 (FIG. 4) depicts the ¹H NMR spectroscopic data of the product obtained in Example 2, according to one or more embodiments provided herein.

### DEFINITIONS

A “catalyst system” is a combination of at least one catalyst compound and at least one activator. When “catalyst system” is used to describe the composition before activation, it comprises the unactivated catalyst complex (precatalyst) together with an activator. When it is used to describe the pair after activation, it comprises the activated complex.

The term “catalyst” refers to a catalyst, a catalyst precursor, a pre-catalyst compound, catalyst compound or a transition metal compound, and these terms are used interchangeably.

The term “catalyst activity” is a measure of how active the catalyst is and is reported as the mass of product (P) produced per mole of catalyst (cat) used (kgP/molcat). For calculating catalyst activity, also referred to as catalyst productivity, only the weight of the transition metal component of the catalyst is used.

Also used herein, Me is methyl, Et is ethyl, Pr is propyl, cPr is cyclopropyl, nPr is n-propyl, iPr is isopropyl, Bu is butyl, nBu is normal butyl, iBu is isobutyl, sBu is sec-butyl, tBu is tert-butyl, Oct is octyl, Ph is phenyl, MAO is methylalumoxane, dme is 1,2-dimethoxyethane, p-tBu is para-tertiary butyl, TMS is trimethylsilyl, TIBAL is triisobutylaluminum, TNOAL is tri(n-octyl)aluminum, p-Me is para-methyl, Bz and Bn are benzyl (i.e., CH₂Ph), THF (also referred to as the is tetrahydrofuran, RT is room temperature (and is 23° C. unless otherwise indicated), tol is toluene, EtOAc is ethyl acetate, Cbz is Carbazole, Cy is cyclohexyl, and MHD is 4-methyl-1,4-hexadiene.

The terms “including” and “comprising” are used in an open-ended fashion, and thus should be interpreted to mean “including, but not limited to.” The phrase “consisting essentially of” means that the described/claimed composition does not include any other components that will materially alter its properties by any more than 5% of that property, and in any case does not include any other component to a level greater than 3 mass %.

The term “or” is intended to encompass both exclusive and inclusive cases, i.e., “A or B” is intended to be synonymous with “at least one of A and B,” unless otherwise expressly specified herein.

The indefinite articles “a” and “an” refer to both singular forms (i.e., “one”) and plural referents (i.e., one or more) unless the context clearly dictates otherwise. For example, embodiments using “an olefin” include embodiments where one, two, or more olefins are used, unless specified to the contrary or the context clearly indicates that only one olefin is used.

### DETAILED DESCRIPTION

This invention relates to the hydroalkenylation of conjugated dienes using a diimine metal complex. The preferred product is a 4 substituted 1,4 hexadiene that is represented by the Formula (XX):

H₂C═C(R^{#})—CH₂—C(R^{#})═CH—CH₃ (XX)

wherein each R^{#} is independently hydrogen, a C₁ to C₂₀ hydrocarbyl group, such as C₁ to C₂₀ alkyl group (alternately a C₁ to C₁₂ alkyl group, such as such as methyl, ethyl, propyl, butyl, pentyl, hexyl, heptyl, octyl, nonyl, decyl, undecyl, dodecyl, or an isomer thereof, such as n-isomers thereof), and/or a C₆ to C₂₀ aryl group (such as phenyl, benzyl naphthyl, styryl, xylyl or an isomer) or substituted variant thereof. A particularly preferred product is 4-methyl-1,4-hexadiene or “MHD”.

In at least one embodiment, the diimine metal complex is a transition metal complex represented by Formula (A):

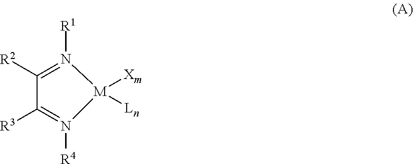

wherein: M is Cr[II], Cr[III], Mn[II], Mn[III], Mn[IV], Fan Fe[III], Ru[II], Ru[III], Ru[IV], Co[II], Co[III], Rh[II], Rh[III], Ni[II], Pd[II], Cu[I], or Cu[II]; X represents an atom or group covalently or ionically bonded to the transition metal M; L is a group datively bound to M; R2, R3, are each independently selected from hydrogen, halogen, hydrocarbyl, substituted hydrocarbyl, heterohydrocarbyl, substituted heterohydrocarbyl or SiR′3 where each R′ is independently selected from hydrogen, halogen, hydrocarbyl, substituted hydrocarbyl, heterohydrocarbyl and substituted heterohydrocarbyl; R2 and R3 may be joined together to form a ring; R1 and R4 are each independently selected from a substituted hydrocarbyl, unsubstituted hydrocarbyl, substituted heterocyclic, or unsubstituted heterocyclic, saturated or unsaturated ring, where the ring has 5, 6, 7, or 8 ring atoms and where substitutions on the ring can join to form additional rings; n is from 0 to 5; m is 1 to 3. Preferably M is Fe.

Specific examples of preferred diimine complexes represented by Formula (A) are as follows:

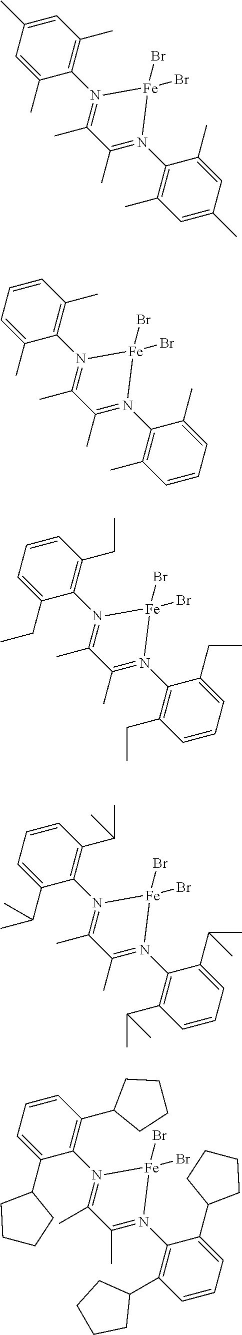

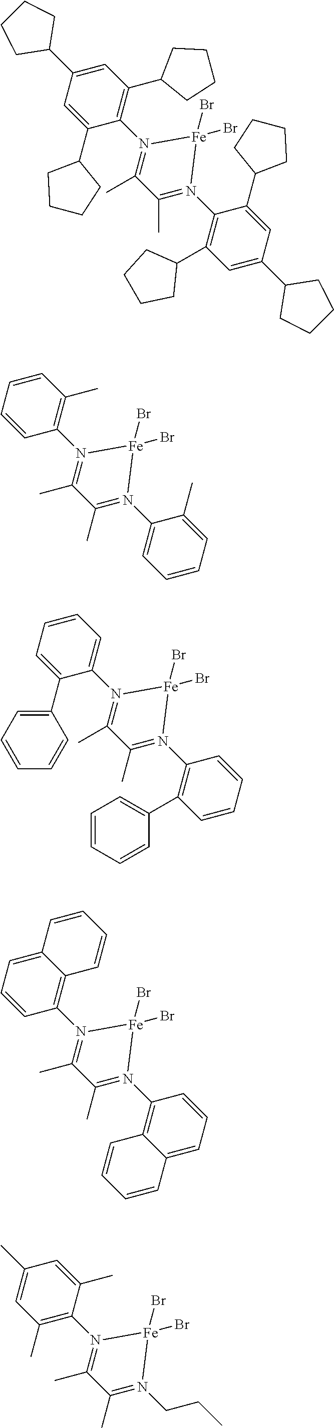

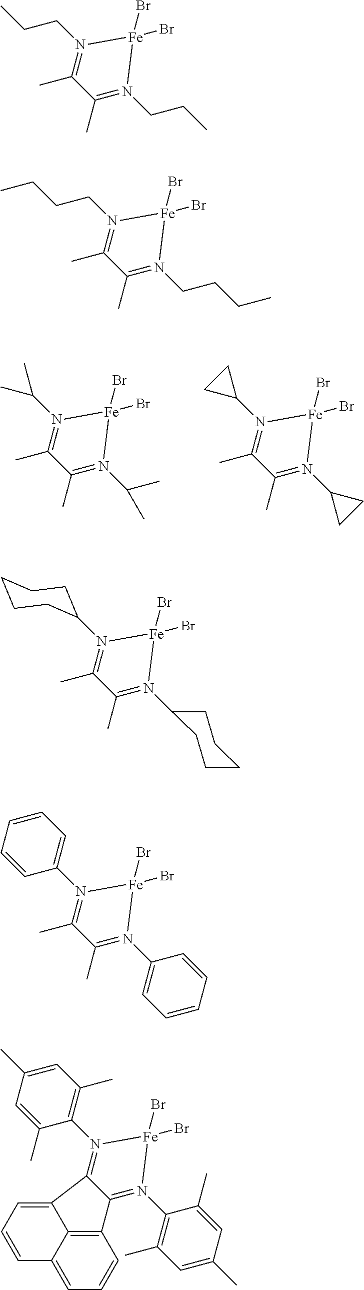

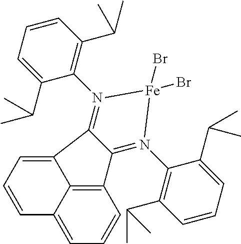

In certain embodiments, the catalyst system includes one or more diimine catalyst complexes described above, and one or more cocatalysts or activators. The terms “cocatalyst” and “activator” are used herein interchangeably and refer to any compound which can activate the diimine catalyst described above by converting the neutral metal compound to the catalytically active metal compound. Suitable activators include Mg(butadiene)(THF)₂ or a hydrocarbylmagnesium halide. The one or more catalyst components of Formula (A) can be combined with one or more activators in any manner known from the literature.

The catalyst systems can also be added to or generated in solution using the conjugated diene reactant as the solvent. Additional solvent, i.e. liquids other than the susbstrate(s) for the hydroalkenylation, are not required but could be used if desired.

The diimine metal complex can be used for the hydroalkenylation of one or more conjugated dienes as one reactant (i.e. a first reactant) and using ethylene and/or any one or more other alpha olefins as another reactant (i.e. a second reactant). Besides ethylene, suitable other alpha olefins include olefins those having 3 to 20 carbon atoms; 3 to 16; or 3 to 12. For example, the other alpha olefins can have anywhere from from 3, 4, or 6 carbon atoms to 8, 12 or 20. A preferred conjugated diene is a diene with 1,3 conjugation.

It has been surprisingly discovered that that the diimine catalyst complex can be synthesized at temperatures greater than −60° C. It has also been surprisingly discovered that the diimine catalyst complex can be synthesized at temperatures greater than −60° in neat diene (i.e. no solvent addition). Indeed, it has been surprisingly discovered that the liquid 1,3-diene reactants are effective solvents for the diimine catalyst system and high catalyst activity with good selectivity are achieved without the use of added solvent. By eliminating additional solvent, the purification of the hydroalkenylation products is simplified and can be achieved by simple thermal separation. The catalyst preparation and hydroalkenylation process is further described with reference to the following non-limiting examples.

### EXAMPLES

Unless otherwise stated, materials were handled using standard chemical techniques. All potentially air-sensitive materials (i.e. catalysts, activators) were manipulated under anhydrous conditions with dry dinitrogen. Reagent grade or better starting materials were purchased from commercial vendors and used as received or purified according to standard procedures. Commercially sourced materials were utilized as received or purified according to standard procedures (Anarego, W. L.; Chair, C. L. Purification of Laboratory Chemicals; 5 ed.; Elsevier: Oxford, 2003). All monomers were subjected to standard procedures to dry and degass the materials. NMR data for catalysts and chemical precursors were recorded on Bruker 400 MHz and 500 MHz NMR Spectrometers. ¹H and ¹³C{¹H} chemical shifts are reported in ppm relative to SiMe₄ (¹H and ¹³C{¹H} δ=0.0 ppm) using residual protio resonances.

### Synthesis of Mg(Butadiene)(THF)₂

Butadiene (100 g) was condensed at −30° C. and then vacuum transferred to a thick-walled flask containing calcium hydride powder. The mixture was allowed to stir for 24 h at −30° C. The butadiene was then cold filtered (˜30° C.) through a chilled, glass fritted funnel packed with calcium hydride.

A 500 mL thick-walled flask was fitted with a PTFE-coated magnetic stirbar. It was then charged with magnesium filings (˜24 g), THF (300 mL), and an alliqout of iodobenzene (˜0.5 mL). The mixture was cooled to −60° C. using cold-bath. Liquid butadiene (25 mL) was then added. The flask was sealed and allowed to warm to room temperature to react while stirring. After 16 hours, a pale yellow suspension had formed. At this time, the flask was opened and additional liquid butadiene (˜30 mL) was quickly added along with THF (100 mL). The additional THF was added to ensure efficient mixing of the the, now thick, suspension. After an additional 18 hours, the flask was unsealed and the reaction mixture mixture passed through a successive series of aluminum mesh sieves to remove unreacted magnesium particulates. Additional THF (˜200 mL) was used to help carry the product through the mesh. Final mesh size was 0.025 inches. The sieved suspension (i.e. filtrate) was collected and filtered through a medium porosity glass fritted funnel. This afforded a pale-yellow/off-white solid that was washed with THF (100 mL) and Et₂O (3×200 mL). Washes were continued until the filtrate ran colorless. The filter cake was then dried under reduced pressure (200 mTorr) for several hours. This afforded 90.01 g of the desired product which was used without further purification. ATR-IR (powder): 2955 (m), 2885 (m), 2846 (m), 2785 (w), 2750 (w), 1579 (m), 1552 (w, sh), 1456 (w), 1404 (w), 1358 (m), 1340 (w), 1244 (vw), 1159 (m), 1027 (s), 943 (m), 899 (m, sh), 870 (vs), 739 (s), 698 (sh), 650 (s), 547 (s) 520 (vs), 502 (vs), 457 (s) cm⁻¹.

### General Synthesis of Diimine Ligands:

A round-bottom flask was fitted with a PTFE coated stir-bar and di-ketone (1.0 eq), amine (2-3 eq), solvent (0.5 M) and 5-10 mol % acid were added. The reaction was allowed to progress at ambient to refluxing conditions for 6-48 hours. Upon completion, the reaction was concentrated and then either rinsed with cold acetone to give the desired diimines as bright yellow or orange crystalline solids in high purity, or in the case of the lower molecular weight diimines, distilled to give desired product as a pale yellow oil.

### General Synthesis for Iron (II) Dihalide Catalysts:

In a round-bottom flask under aerobic conditions, iron (II) dihalide (1 eq) was combined with tetrahydrofuran (0.1 M) in the presence of a magnetic stir-bar. Diimine ligand (0.8-1.2 eq) was added to the stirring solution in a single portion at ambient to 50° C. and allowed to react for 4-48 hours. Upon significant color change, the resulting mixture was then concentrated to a solid under reduced pressure. The solids were suspended in diethyl ether and filtered. The filter cake was washed with 3-5× additional portions of diethyl ether and dried under reduced pressure.

### Preparation of N,N-dimesitylbutane-2,3-diimine (^{Mes}DI)

A 500 mL round-bottom flask was fitted with a PTFE coated stir-bar and Dean Stark apparatus. The flask was charged with 2,3-butanedione (15.00 g, 0.174 mol), 2,4-6-trimethylaniline (49.36 g, 0.365 mol, 2.095 equiv), toluene (400 mL) and a catalytic quantity of para-toluenesulfonic acid (0.5 g). The reaction flask atmosphere was then purged with nitrogen and the mixture heated to 80° C. for 4 hours. The temperature was first held at 80° C. to form the monosubstituted imine-one, to reduce the quantity of 2,3-butanedione which could condense in the Dean Stark. After 4 hours, the reaction mixture was brought to reflux and allowed to react for 36 hours; during which time 6.1 mL of water was collected in the Dean Stark. The reaction mixture was then cooled to room temperature and reduced in volume under a gentle nitrogen flow to remove volatiles. Crystalline ^{Mes}DI grew from the non-volatile brown reaction mixture over ˜3 hours. Chilled (0-5° C.) acetone was then added (200 mL) and the suspension filtered. This afforded a filter-cake of crystalline ^{Mes}DI which was washed with additional portions of chilled acetone until bright yellow. Yield 45.1 g, 88.83% ¹H NMR (400.1 MHz, benzene-d₆, 20° C.): δ=6.84 (s, 4H, o-Mes), 2.23 (s, 6H, p-Mes) ppm. ¹³C{¹H} NMR (120.2 MHz, benzene-d₆, 20° C.): δ=168.4, 146.8, 132.4, 129.1, 124.6, 20.9, 18.0, 15.8 ppm.

### Preparation of N,N-dimesitylbutane-2,3-diimine iron(II) dibromide (^{Mes}DIFeBr₂)

In a 1 liter round-bottom flask, iron(II) dibromide (14.13 g, 65.5 mmol) was combined with tetrahydrofuran (800 mL) in the presence of a magnetic stir-bar. ^{Mes}DI (20.00 g, 62.38 mmol) was added as a solid in a single portion. The resulting mixture was heated to 50° C. and allowed to react for 24 hours. The resulting mixture was then concentrated to a solid under reduced pressure. The solid was then suspended in Et₂O (200 mL) and filtered. The maroon colored filter cake was washed with addition portions of Et₂O (3×200 mL) then dried under reduced pressure (200 mTorr). The product is maroon in the solid-state and green in THF solution. Yield: 31.00 g, 92.65% ¹H NMR (400.1 MHz, THF-d₈, 20° C.): δ=110.67, 15.24, 8.99, 5.16 ppm.

### Preparation of 4-methyl-1,4-hexadiene (Parr Autoclave)

### Example 1

In a glovebox, a 0.6 L Parr Autoclave fitted with mechanical stirring, temperature control and glass liner was charged with a −60° C. solution of isoprene (50.8 g, 75 mL) and ^{Mes}DIFeBr₂ (0.400 g, 20 mL). The mixture was stirred for 20 minutes. Thereafter, Mg(Butadiene)*THF₂ (0.199 g) was added. No alkanes or other solvents were added to this catalyst solution. The Parr reactor was then sealed, and connected to cooling, HMI interface and ethylene gas supply. During this time (— 20 minutes) the temperature of the reactor contents gradually rose to 14° C. The unit was then pressurized to 700 PSIG ethylene. Upon addition of ethylene, the reaction appeared to initiate as indicated by an appreciable exotherm. With active cooing, the reactor temperature rose from room temperature to nearly 50° C. After 40 min at 50° C., the reactor was vented. The liquid contents were filtered through basic alumina to remove catalyst reside.

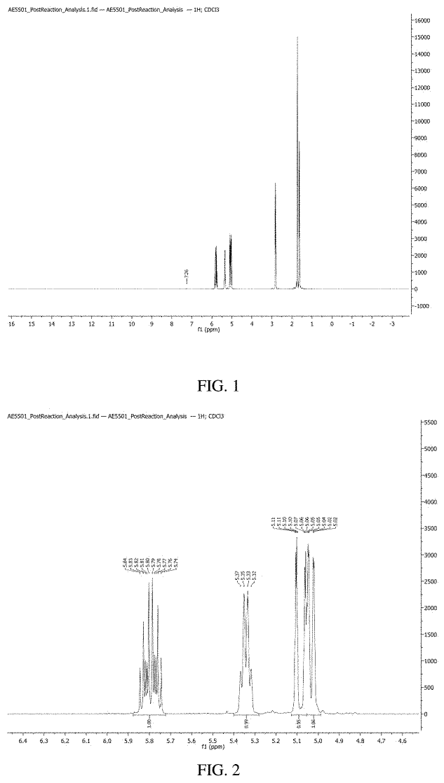

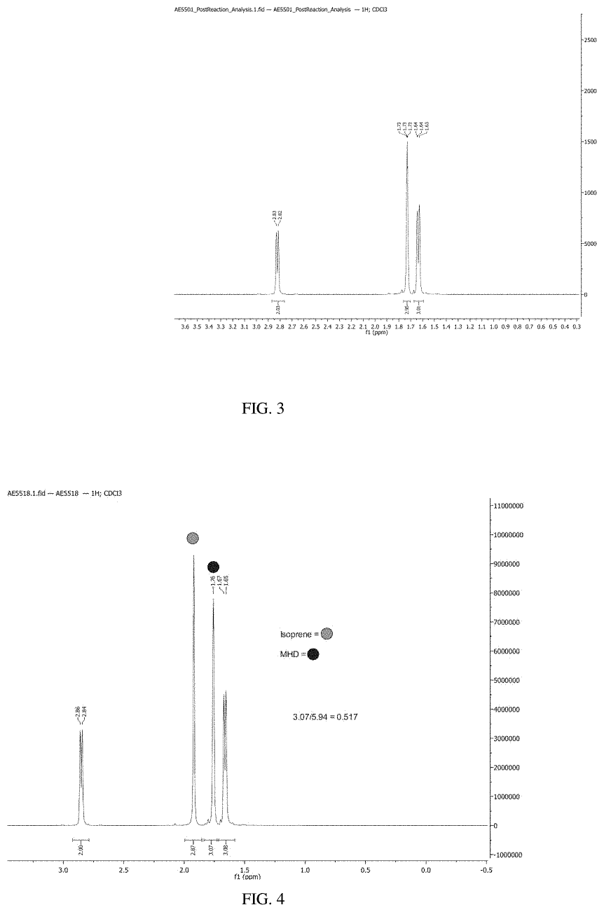

A colorless liquid was obtained and was subjected to ¹H and ¹³C{1H} NMR analysis. NMR spectroscopic data (C6D6; 1H) showed full conversion of isoprene (50.8 g) to (z)-4-methyl-1,4-hexadiene, as depicted in FIGS. 1-3.

Crude product (post filtration) was extremely clean. Full conversion, near complete selectivity. A lower limit to the TOF was calculated as follows: TOF=(mol isoprene)*(1/mol cat)*(1/time)=(1499.6 mmol)*(1/0.222 mmol)*(1/0.75 h)=9006 h⁻¹.

### Example 2

In a glovebox, two 0.6 L Parr Autoclaves fitted with mechanical stirring, temperature control and glass liner were charged at room temperature (about 23° C.) with isoprene (204 g, 300 mL). Then to each reactor, ^{Mes}DIFeBr₂ (0.118 g, 20 mL) was added as a solid in one portion. [Utilized 0.01 mol % catalyst loading (mg[cat]/kg[isoprene]=(118 mg/0.204 Kg)=578 ppm.] Thereafter, Mg(Butadiene)*THF₂ (0.199 g) was added as a solid in one portion. The materials were not mixed. No alkanes or other solvents were added to this catalyst solution. The Parr reactors were then sealed, and connected to cooling, HMI interface and gas supply. The parallel units were then pressurized to 500 PSIG ethylene. Upon addition of ethylene, the reaction appeared to initiate as indicated by an exotherm. The reactor was then heated to 45° C. and allowed to react for 90 min. The reactors were then cooled to RT, vented and opened. The reactor contents were combined, filtered through alumina and analyzed by ¹H NMR spectroscopy. Integration showed the composition to be 51.7 MHD with balance isoprene (see FIG. 4). Materials were then purified by distillation (BP at 1 atm: Isoprene—˜35-37° C.; MHD-91° C.) to afford pure MHD. The crude ¹H NMR spectrum is shown in FIG. 4. No other by-products were observed.

### Example 3

In a glovebox, to a 20 mL vial was added Mg(Butadiene)*THF₂ (220 mg) and 10 mL of isoprene; the resulting mixture was stirred at −30° C. After 20 minutes, N,N-dimesitylbutane-2,3-diimine iron(II) dichloride (^{Mes}DIFeCl₂) (380 mg) was then added as a solid in a single portion. The mixture was allowed to reach room temperature and then loaded into a pressure vessel, rinsing the vial with an additional 30 mL of isoprene.

In a 2 L autoclave reactor, isoprene (1100 mL) was injected under a nitrogen atmosphere and the catalyst pressure vessel attached. The reactor body was heated to 40° C. and using a stream of ethylene at a 2.5 SLM, the catalyst mixture was injected. The reactor was then set to reach a pressure of 400 PSI, which it did within 5 minutes. The reactor underwent an intense exotherm, but returned to the set temperature of 50° C. and was allowed to react for 60 minutes. After 60 minutes, the reactor was then cooled to RT, vented and opened. The light yellow liquid contents were filtered through alumina, removing spent catalyst, to give a colorless filtrate.

The filtrate was subjected to ¹H and ¹³C{1H} NMR analysis. NMR spectroscopic data (CDCl₃; 1H) showed full conversion of isoprene to (z)-4-methyl-1,4-hexadiene. 1000 g of product was obtained, giving a yield of 92.6%, and a TOF=13,200 h⁻¹.

### Example 4

In a glovebox, to a 20 mL vial was added Mg(Butadiene)*THF₂ (285 mg) and 20 mL of isoprene; the resulting mixture was stirred at −30° C. After 20 minutes, N,N-bis(2,6-diisopropylphenyl)acenaphthylene-1,2-diimine iron(II) dibromide (dippBIANFeBr₂) (895 mg) was then added as a solid in a single portion. The mixture was allowed to reach room temperature and then loaded into a pressure vessel, rinsing the vial with an additional 30 mL of isoprene.

In a 2 L autoclave, isoprene (500 mL) was injected under a nitrogen atmosphere and the catalyst pressure vessel attached. The reactor body was heated to 40° C., and using a stream of ethylene at a 10 SLM, the catalyst mixture was injected. The reactor was then set to reach a pressure of 400 PSI and 50° C., which it did within 5 minutes. After 180 minutes, the reactor was then cooled to RT, vented and opened. The light yellow liquid contents were filtered through alumina, removing spent catalyst, to give a colorless filtrate.

The filtrate was subjected to ¹H and ¹³C{1H} NMR analysis. NMR spectroscopic data (CDCl₃; 1H) showed 60% conversion of isoprene to (z)-4-methyl-1,4-hexadiene, with the balance being isoprene, no other products were observed.

### Example 5

In a glovebox, to a 20 mL vial was added Mg(Butadiene)*THF₂ (220 mg) and 20 mL of isoprene; the resulting mixture was stirred at −30° C. After 20 minutes, N,N-diphenylbutane-2,3-diimine iron(II) dibromide (PhDIFeBr₂) (370 mg) was then added as a solid in a single portion. The mixture was allowed to reach room temperature and then loaded into a pressure vessel, rinsing the vial with an additional 30 mL of isoprene.

In a 2 L autoclave, isoprene (500 mL) was injected under a nitrogen atmosphere and the catalyst pressure vessel attached. The reactor body was heated to 40° C., and using a stream of ethylene at a 10 SLM, the catalyst mixture was injected. The reactor was then set to reach a pressure of 400 PSI and 50° C., which it did within 25 minutes. After 65 minutes, the reactor was then cooled to RT, vented and opened. The light yellow liquid contents were filtered through alumina, removing spent catalyst, to give a colorless filtrate.

The filtrate was subjected to ¹H and ¹³C{1H} NMR analysis. NMR spectroscopic data (CDCl₃; 1H) showed full conversion of isoprene to a mixture containing (z)-4-methyl-1,4-hexadiene and (E)-2-methylhexa-1,4-diene, no residual isoprene was observed.

### Example 6

In a glovebox, to a 20 mL vial was added Mg(Butadiene)*THF₂ (200 mg) and 20 mL of isoprene; the resulting mixture was stirred at −30° C. After 10 minutes, ^{Mes}DIFeBr₂ (380 mg) was then added as a solid in a single portion. The mixture was allowed to reach room temperature and then loaded into a pressure vessel, rinsing the vial with an additional 10 mL of isoprene.

In a 2 L autoclave, isoprene (1,100 mL) was injected under a nitrogen atmosphere and the catalyst pressure vessel attached. The reactor body was heated to 40° C., and using a stream of ethylene at a 2.5 SLM, the catalyst mixture was injected. The reactor was then set to reach a pressure of 400 PSI and 50° C., which it did within 2 minutes. After 45 minutes, the reactor was then cooled to RT, vented and opened. The light yellow liquid contents were filtered through alumina, removing spent catalyst, to give a colorless filtrate.

The filtrate was subjected to ¹H and ¹³C{1H} NMR analysis. NMR spectroscopic data (CDCl₃; 1H) showed full conversion of isoprene to (z)-4-methyl-1,4-hexadiene. 988 g of desired material was collected giving a yield of 91.5% and at TOF=18,900 h⁻¹.

### Example 7

In a glovebox, to a 20 mL vial was added Mg(Butadiene)*THF₂ (220 mg) and 20 mL of isoprene; the resulting mixture was stirred at 0° C. After 10 minutes, N-mesityl-N-propylbutane-2,3-diimine iron(II) dibromide (^{Mes Propyl}DIFeBr₂) (160 mg) was then added as a solid in a single portion. The mixture was allowed to reach room temperature and then loaded into a pressure vessel, rinsing the vial with an additional 10 mL of isoprene.

In a 2 L autoclave, isoprene (500 mL) was injected under a nitrogen atmosphere and the catalyst pressure vessel attached. The reactor body was heated to 40° C., and using a stream of ethylene at a 10 SLM, the catalyst mixture was injected. The reactor was then set to reach a pressure of 400 PSI and 50° C., which it did within 15 minutes. After 120 minutes the reactor was then cooled to RT, vented and opened. The light yellow liquid contents were filtered through alumina, removing spent catalyst, to give a colorless filtrate.

The filtrate was subjected to ¹H and ¹³C{1H} NMR analysis. NMR spectroscopic data (CDCl₃; 1H) showed hydrovinylation products and 18% isoprene.

### Example 8

In a glovebox, to a 20 mL vial was added Mg(Butadiene)*THF₂ (610 mg) and 20 mL of isoprene; the resulting mixture was stirred at −20° C. After 10 minutes, N,N-dicyclohexylbutane-2,3-diimine iron(II) dibromide (^{Cyclohexyl}DIFeBr₂) (1150 mg) was then added as a solid in a single portion. The mixture was allowed to reach room temperature and then loaded into a pressure vessel, rinsing the vial with an additional 10 mL of isoprene.

In a 2 L autoclave, isoprene (500 mL) was injected under a nitrogen atmosphere and the catalyst pressure vessel attached. The reactor body was heated to 35° C., and using a stream of ethylene at a 10 SLM, the catalyst mixture was injected. The reactor was then set to reach a pressure of 400 PSI and 50° C., which it did in approximately 5 minutes. After 66 minutes, the reactor was then cooled to RT, vented and opened. The light yellow liquid contents were filtered through alumina, removing spent catalyst, to give a colorless filtrate.

The filtrate was subjected to ¹H and ¹³C1{1H} NMR analysis. NMR spectroscopic data (CDCl₃; 1H) showed 30% conversion to (z)-4-methyl-1,4-hexadiene with the balance being isoprene.

### Example 9

In a glovebox, to a 20 mL vial was added Mg(Butadiene)*THF₂ (278 mg) and 20 mL of isoprene; the resulting mixture was stirred at −20° C. After 10 minutes, N,N-bis(mesityl)acenaphthylene-1,2-diimine iron(II) dibromide (MesBIANFeBr₂) (790 mg) was then added as a solid in a single portion. The mixture was allowed to reach room temperature and then loaded into a pressure vessel, rinsing the vial with an additional 10 mL of isoprene.

In a 2 L autoclave, isoprene (500 mL) was injected under a nitrogen atmosphere and the catalyst pressure vessel attached. The reactor body was heated to 30° C., where using a stream of ethylene at a 10 SLM the catalyst mixture was injected. The reactor was then set to reach a pressure of 400 PSI and 50° C., which it did in approximately 10 minutes. After 120 minutes the reactor was then cooled to RT, vented and opened. The light yellow liquid contents were filtered through alumina, removing spent catalyst, to give a colorless filtrate.

The filtrate was subjected to ¹H and ¹³C1{1H} NMR analysis. NMR spectroscopic data (CDCl₃; 1H) showed 20% conversion to (z)-4-methyl-1,4-hexadiene with the balance being isoprene.

### Example 10

In a glovebox, methylmagnesium chloride as a 3.0 M solution in THF (0.20 mL) was stirred with 10 mL of isoprene at −20° C. After 10 minutes, ^{Mes}DIFeBr₂ (150 mg) was then added as a solid in a single portion. The mixture was allowed to reach room temperature and then loaded into a pressure vessel, rinsing the vial with an additional 10 mL of isoprene.

In a 2 L autoclave, isoprene (1,000 mL measured by sight glass) was injected under a nitrogen atmosphere and the catalyst pressure vessel attached. The reactor body was heated to 40° C., and using a stream of ethylene at a 2.5 SLM, the catalyst mixture was injected. The reactor was then set to reach a pressure of 400 PSI and 50° C., which it did within 4 minutes. After 69 minutes, the reactor was then cooled to RT, vented and opened. The light yellow liquid contents were filtered through alumina, removing spent catalyst, to give a colorless filtrate.

The filtrate was subjected to ¹H NMR analysis. NMR spectroscopic data (CDCl₃; 1H) showed full conversion of isoprene to (z)-4-methyl-1,4-hexadiene. 718 g of desired material was collected giving a yield of 70.2% and at TOF=22,300 h⁻¹.

### Example 11

In a glovebox, methylmagnesium chloride as a 3.0 M solution in THF (0.20 mL) was stirred with 10 mL of isoprene at 23° C. After 5 minutes, ^{Mes}DIFeBr₂ (150 mg) was then added as a solid in a single portion. The mixture was allowed to stir and then loaded into a pressure vessel, rinsing the vial with an additional 20 mL of isoprene.

In a 2 L autoclave, isoprene (1,000 mL measured by sight glass) was injected under a nitrogen atmosphere and the catalyst pressure vessel attached. The reactor body was heated to 40° C., and using a stream of ethylene at a 2.5 SLM, the catalyst mixture was injected. The reactor was then set to reach a pressure of 400 PSI and 50° C., which it did within 4 minutes. After 56 minutes, the reactor was then cooled to RT, vented and opened. The light yellow liquid contents were filtered through alumina, and silica gel removing spent catalyst, to give a colorless filtrate.

The filtrate was subjected to ¹H NMR analysis. NMR spectroscopic data (CDCl₃; 1H) showed full conversion of isoprene to (z)-4-methyl-1,4-hexadiene. 773 g of desired material was collected giving a yield of 80.36% and at TOF=29,000 h⁻¹.

In view of the foregoing, it was discovered that an improved method was found to be effective whereby active catalyst could be generated at temperatures between −60° C. and room temperature in neat diene. Catalyst performance was determined to be inversely correlated with activation temperature. Some catalyst performance debits were observed with room temperature catalyst activation. However, selectivity was shown to remain excellent.

While the crude ^{Mes}DIFe(COD) material was active, it was operationally inconvenient to handle in solid form due to tacky physical properties. An operationally convenient powder form of this catalyst was accessible by concentrating an alkane solution of ^{Mes}DIFe(COD) to a solid in the presence of Celite to afford a flowable powder.

It was further recognized that liquid 1,3-diene monomers are effective solvents for the catalyst system and it was shown that high catalyst activity and good selectivity could be achieved without the use of solvent. By eliminating solvent, the purification of the hydrovinylation products was simplified to the mere thermal separation of the liquid products from the liquid starting materials if any remained (e.g., separation of isoprene from (z)-4-methyl-1-4-hexadiene).

It was further discovered that in the absence of solvent, the high concentration of conjugated diene at the end of a batch run could lead to re-incorporation side products. It was found that sufficient quantities of alpha olefin must be dissolved in the conjugated diene in order to achieve higher selectivity. When using ethylene as an alpha olefin, using high pressure ethylene eliminates this as a concern. Ethylene pressures of greater than 300 PSIG, and more preferably greater than 400, 500, 600, or 700 PSIG can be used. In some embodiments, the pressure in any reactor used herein can be 0.1 to 100 atm, e.g., 0.5 to 75 atm or 1 to 50 atm. Alternatively, the pressure in any reactor used herein can be 1 to 50,000 atm, e.g., 1 to 25,000 atm.

In other embodiments, the present invention relates to the following numbered paragraphs:

1. A method for making a catalyst useful for making substituted diene monomers, comprising:

mixing a conjugated diene having at least five carbon atoms, an iron diimine complex, and an activator at a temperature of about −60° C. to about 23° C. to provide a catalyst solution; and
introducing one or more alpha olefins to obtain a product comprising the substituted diene monomer.

2. The method according to paragraph 1, wherein no alkane solvent is used to make the catalyst solution and the catalyst solution consists essentially of the diene, the iron diimine complex, and the activator.

3. The method according to paragraph 1 or 2, wherein no alkane solvent is used to make the catalyst solution and the catalyst solution consists of the diene, the iron diimine complex, and the activator.

4. The method according to any paragraph 1 to 3, wherein the substituted diene monomer comprises 4 substituted 1,4 hexadiene.

5. The method according to any paragraph 1 to 4, wherein the substituted diene monomer is 4 substituted 1,4 hexadiene.

6. The method according to any paragraph 1 to 5, wherein the one or more alpha olefins is ethylene.

7. A method for making 4 substituted 1,4 hexadiene, comprising:

mixing a conjugated diene having at least five carbon atoms, an iron diimine complex, and an activator at a temperature of about −60° C. to about 23° C. to provide a catalyst solution; and

introducing one or more alpha olefins to obtain a monomer comprising the 4 substituted 1,4 hexadiene.

8. The method according to paragraph 7, wherein the one or more alpha olefins comprises ethylene at a pressure of at least 300 psig.

9. The method according to paragraph 7 or 8, wherein no alkane solvent is used to make the catalyst solution and the catalyst solution consists essentially of the diene, the iron diimine complex, and an activator.

10. The method according to any paragraph 7 to 9, wherein the iron diimine complex is represented by the structure:

wherein:

M is Fe;

X represents an atom or group covalently or ionically bonded to the transition metal M;

L is a group datively bound to M;

R2, R3, are each independently selected from hydrogen, halogen, hydrocarbyl, substituted hydrocarbyl, heterohydrocarbyl, substituted heterohydrocarbyl or SiR′3 where each R′ is independently selected from hydrogen, halogen, hydrocarbyl, substituted hydrocarbyl, heterohydrocarbyl and substituted heterohydrocarbyl;

R1 and R4 are each independently selected from a substituted hydrocarbyl, unsubstituted hydrocarbyl, substituted heterocyclic, or unsubstituted heterocyclic, saturated or unsaturated ring, where the ring has 5, 6, 7, or 8 ring atoms and where substitutions on the ring can join to form additional rings;

n is from 0 to 5; and

m is 1 to 3.

11. The method according to any paragraph 7 to 10, wherein the activator is Mg(butadiene)(THF)₂ or a hydrocarbylmagnesium halide.

Certain embodiments and features have been described using a set of numerical upper limits and a set of numerical lower limits. It should be appreciated that ranges from any lower limit to any upper limit are contemplated unless otherwise indicated. Certain lower limits, upper limits and ranges appear in one or more claims below. All numerical values are “about” or “approximately” the indicated value, and take into account experimental error and variations that would be expected by a person having ordinary skill in the art.

Various terms have been defined above. To the extent a term used in a claim is not defined above, it should be given the broadest definition persons in the pertinent art have given that term as reflected in at least one printed publication or issued patent. Further, all documents described herein are incorporated by reference herein, including any priority documents and/or testing procedures to the extent they are not inconsistent with this text. As is apparent from the foregoing general description and the specific embodiments, while forms of the invention have been illustrated and described, various modifications can be made without departing from the spirit and scope of the invention. Accordingly, it is not intended that the invention be limited thereby. Likewise, the term “comprising” is considered synonymous with the term “including.” Likewise whenever a composition, an element or a group of elements is preceded with the transitional phrase “comprising”, it is understood that we also contemplate the same composition or group of elements with transitional phrases “consisting essentially of,” “consisting of”, “selected from the group of consisting of,” or “is” preceding the recitation of the composition, element, or elements and vice versa.

While the foregoing is directed to embodiments of the present invention, other and further embodiments of the invention may be devised without departing from the basic scope thereof, and the scope thereof is determined by the claims that follow.

What is claimed is:

1. A method for making a catalyst useful for making substituted diene monomers, comprising:
mixing a conjugated diene having at least five carbon atoms, an iron diimine complex, and an activator at a temperature of about −60° C. to about 23° C. to provide a catalyst solution; and
introducing one or more alpha olefins to obtain a product comprising the substituted diene monomer.

2. The method of claim 1, wherein no alkane solvent is used to make the catalyst solution and the catalyst solution consists essentially of the diene, the iron diimine complex, and the activator.

3. The method of claim 1, wherein no alkane solvent is used to make the catalyst solution and the catalyst solution consists of the diene, the iron diimine complex, and the activator.

4. The method of claim 1, wherein the substituted diene monomer comprises 4 substituted 1,4 hexadiene.

5. The method of claim 1, wherein the substituted diene monomer is 4 substituted 1,4 hexadiene.

6. The method of claim 1, wherein the one or more alpha olefins is ethylene.

7. A method for making 4 substituted 1,4 hexadiene, comprising:
mixing a conjugated diene having at least five carbon atoms, an iron diimine complex, and an activator at a temperature of about −60° C. to about 23° C. to provide a catalyst solution; and
introducing one or more alpha olefins to obtain a monomer comprising the 4 substituted 1,4 hexadiene.

8. The method of claim 7, wherein the one or more alpha olefins comprises ethylene at a pressure of at least 300 psig.

9. The method of claim 8, wherein no alkane solvent is used to make the catalyst solution and the catalyst solution consists essentially of the diene, the iron diimine complex, and an activator.

10. The method of claim 7, wherein the iron diimine complex is represented by the structure: 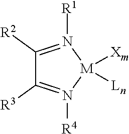
wherein:
M is Fe;
X represents an atom or group covalently or ionically bonded to the transition metal M;
L is a group datively bound to M;
R2, R3, are each independently selected from hydrogen, halogen, hydrocarbyl, substituted hydrocarbyl, heterohydrocarbyl, substituted heterohydrocarbyl or SiR′3 where each R′ is independently selected from hydrogen, halogen, hydrocarbyl, substituted hydrocarbyl, heterohydrocarbyl and substituted heterohydrocarbyl;
R1 and R4 are each independently selected from a substituted hydrocarbyl, unsubstituted hydrocarbyl, substituted heterocyclic, or unsubstituted heterocyclic, saturated or unsaturated ring, where the ring has 5, 6, 7, or 8 ring atoms and where substitutions on the ring can join to form additional rings;
n is from 0 to 5; and
m is 1 to 3.

11. The method of claim 7, wherein the activator is Mg(butadiene)(THF)₂ or a hydrocarbylmagnesium halide.
